(19) United States
(12) Patent Application Publication
(10) Pub. No.: US 2021/0140932 A1
(43) Pub. Date: May 13, 2021

(54) DETECTION OF GASES AND VAPORS BY PATTERNED NANOPARTICLE LIQUID CRYSTAL ALIGNMENT

(71) Applicant: KENT STATE UNIVERSITY, Kent, OH (US)

(72) Inventors: Torsten HEGMANN, Kent, OH (US); Elda HEGMANN, Kent, OH (US); Marianne PRÉVÔT, Kent, OH (US); Ahlam NEMATI, Kent, OH (US); Julie VANEGAS GARCIA, Kent, OH (US)

(21) Appl. No.: 17/123,337

(22) Filed: Dec. 16, 2020

Related U.S. Application Data
(62) Division of application No. 16/640,387, filed on Feb. 20, 2020.
(60) Provisional application No. 62/586,304, filed on Nov. 15, 2017.

(51) Int. Cl. G01N 33/00; G01N 27/12; G02F 1/1337

(52) U.S. Cl. CPC G01N 33/0047; G02F 1/133711; G01N 27/126

(57) ABSTRACT

A sensor for detecting non-hazardous and especially hazardous gases and/or vapors comprises a liquid crystal cell generally having a standard substrate and a conductive electrode layer thereon. An alignment layer is desirably located on the electrode layer and contains one or more types of metal nanoparticles that cover at least a portion of the alignment layer. The nanoparticles contain at least one type of ligand thereon that is capable of sensing a specific type of non-hazardous or hazardous gas. The sensor is very sensitive and can detect the gases or vapors contained within air, or the like, up to 1 part per million.

### FIELD OF THE INVENTION

A sensor, for detecting non-hazardous and especially hazardous gas and/or vapor compounds, has a liquid crystal cell comprising standard elements such as a substrate that can be glass, quartz, or a polymer, and a conductive electrode layer deposit thereon such as indium oxide, tin oxide, or indium tin oxide. An alignment layer resides on the electrode layer and contains one or more types of metal nanoparticles that generally cover the alignment layer (homogeneous or homeotropic), thereby creating any specific or arbitrary pattern, symbol, design, etc. The alignment layer can be a polyimide, a polyvinyl alcohol, SiOₓ, and the like. The nanoparticles contain at least one type of ligand thereon that is capable of chemically reacting with a specific type of non-hazardous or hazardous gas. The sensor is very sensitive and can detect the gases or vapors contained within air, or the like, as low as 1 part per million.

### BACKGROUND OF THE INVENTION

Liquid crystals (LCs) have assumed their place as one of the most important materials of the information age. LC displays (LCDs) play a significant role in our everyday life; from handheld personal devices to professional applications and large-panel LCD TVs. LCs are liquids possessing long-range orientational ordering, lacking in most instances (phases) long-range positional ordering of the constituent molecules. In the case of the one-dimensionally ordered fluid nematic phase used in most display applications, intrinsic elastic interactions align the LC molecules along some preferred direction (director), which in most cases forms the optical axis of the material. Typically, nematic LCs are used in thin films, sandwiched between two glass substrates featuring transparent electrodes (usually indium tin oxide, ITO). These substrates are covered with so-called alignment layers, whose main role is to define the boundary conditions of the director to ensure uniform distribution of the optical axis is the entire LC thin film. These predominant boundary conditions are referred to as “homogeneous” (director lies in the plane of the thin film; usually with a small pre-tilt), “homeotropic” (director is normal to the plane of the thin film) or, less frequently, intermediate “tilted”.

Alignment layers commonly feature some type of anisotropy that induces a preferred orientation for the LC director on the surface. Unidirectionally rubbed polyimides are the most widely used alignment layers, providing stable alignment of nematic and smectic LCs for various display modes. However, this method also has numerous disadvantages, such as polymer debris resulting from the rubbing with a velvet cloth (using rubbing machines) and inhomogeneous, site-dependent contrast ratios in the final display, which can only be avoided by careful monitoring of the manufacturing conditions in clean rooms, see J. van Haaren, Nature 2001, 411, 29. Ion-beam deposition or plasma bombardment of thin polymer, SiNx, diamond-like carbon, or other thin films deposited on substrates are studied as well known to the art and to the literature.

Although these processes have demonstrated their durability and have been implemented in large-scale production environments, they usually require many fabrication steps, high processing temperatures, and sometimes, high vacuum environment.

In addition, many LC applications require patterned alignment of the LC to provide spatial modulation of the optical axis, for example, for the wave front control applications. Usually, in order to obtain patterned alignment, complicated and expensive photolithography techniques must be used. With the use of photoalignment, the process can be significantly simplified, but still requires design and fabrication of photo-masks as well as the deposition of a photosensitive polymer layer using spin coating and baking. Other approaches include micropatterning using a sharp stylus.

The effect of homeotropic alignment of nematic LCs via doping with a small quantity of thiol-capped gold nanoparticles (NPs) has recently been demonstrated; see H. Qi, B. Kinkead, T. Hegmann, Adv. Funct. Mater. 2008, 18, 212; H. Qi, T. Hegmann, ACS Appl. Mater. Interf. 2009, 1, 1731; and M. Urbanski, B. Kinkead, H. Qi, T. Hegmann, H.-S. Kitzerow, Nanoscale 2010, 2, 1118. The NPs migrate and adsorb to the interface formed between the LC films and the substrate, where they induce homeotropic alignment of the director over the entire area of the cell. A similar effect is achieved if NPs are deposited onto the surface before filling of the test cell with the LC material. This leads to a uniform coverage of the surface with the NPs and, in turn, uniform vertical alignment of the LC over the entire area. The homeotropic anchoring of the LC molecules on the NPs is accompanied by a contrast inversion effect, i.e. under the action of a low-frequency electric field, “dielectrically positive” LCs (Δε>0, the dielectric anisotropy Δε is defined as Δε=ε_{∥}−ε_{⊥}, where ε_{∥} is the dielectric permittivity parallel to the long molecular axis and ε_{⊥} the dielectric permittivity perpendicular to the long molecular axis) effectively act as dielectrically negative nematic LC (Δε<0) and undergoes a transition from the homeotropic to the homogenous state, see H. Qi, B. Kinkead, T. Hegmann, Adv. Funct. Mater. 2008, 18, 212.

### SUMMARY OF THE INVENTION

Toxic, or hazardous, or non-hazardous gas sensor technology based on nanoparticles (NPs) and particularly surface functionalization thereof can induce and/or alter the orientation of nematic liquid crystal molecules in direct contact with them. Applying this concept, nanoparticles such as metals in the size regime between 1 and 20 nm with reactive surface ligands were synthesized and patterned via ink-jet printing or spray painting through stencils to devise unique sensors for multiple hazardous gases comprising halogens, for example bromine, and iodine; phosgene; cyanide; amines; hydrazines; dimethyl sulfide and dimethyl selenium; or less hazardous gases and vapors such as ketones including dialkyl chalcogenides. The combination of NP ink-jet printing (or spray painting through stencils) and electro-optical responses of nematic liquid crystals (N-LCs) in contact with these NPs have been found to yield highly sensitive and selective, non-colorimetric sensors, wherein the sensing event itself produces a direct visual readout or warning without the use of electrical power (i.e. patterned light transmission; Sense-To-Image).

In conjunction with a light source on one side of the sensor device and a patterned photodetector (array) on the other side, these sensors can also be used for remote sensing and as fully analytical sensors providing dose×time analytical data for monitoring gas and vapor concentrations over a given time interval.

Stated differently, nanoparticles and particularly their surface functionalization can induce and alter the orientation of nematic liquid crystal molecules in direct contact with them. Thus, gold nanoparticles in the size regime between about 1 and about 10 nm or about 20 nm with reactive surface ligands can be synthesized and patterned via ink-jet printing to devise unique sensors for multiple hazardous (chlorine, phosgene, cyanide, amines, hydrazine, dimethyl sulfides and dimethyl seleniums, dialkyl chalcogenides) or less hazardous gases and vapors (ketones). The combination of nanoparticle ink-jet printing and established concepts of optical and electro-optical responses of nematic liquid crystals in contact with nanoparticles and other surfaces can create highly sensitive and selective sensors, where the sensing event produces a direct visual readout or warning without the use of electrical power. Hence, the effects of chemical functionalization and chemical reactions on nanoparticle surfaces on the alignment of nematic liquid crystals and the concomitant alteration of their optical and electro-optical responses is set forth herein. That is, liquid crystal sensors are used for the simultaneous quantitative and qualitative detection of multiple toxic and non-toxic gases and vapors. Another advantage of the present invention is that wearable or remote, low or no power sensors that can save the lives of and avoid harm to firefighters, military personnel in conflict zones, and chemists in lab and industry environments. Furthermore, these sensors are highly suitable to monitor disease progression and recession in patients with diabetes, cancer, or liver disease.

The present invention relates to toxic gas and vapor sensors for the qualitative and quantitative detection of toxic gases and vapors. These integrative sensors systems can either display an unmistakable warning image in the presence of toxic gases and vapors without any electrical power or provide ppm-level dose×time data when interfaced with OLED light source and printed organic photodetector (OPD). The active component of the sensors is based on reactive, ink-jet printed nanoparticle alignment layers for nematic liquid crystals. In analogy to omnipresent liquid crystal displays (LCDs), an image (or readable pattern) emerges due to the presence of specific hazardous toxic gases and vapors that could affect the lives and health of firefighters, military personnel in conflict zones, first responders, workers in chemical manufacturing (e.g., gold mining), among others. Sensors for volatile ketones can also be used to monitor disease states and disease progression such as in diabetes, liver disease, or cancer.

It is thus an object of the present invention to provide a highly versatile sensor platform based on nanoparticle-induced liquid crystal (LC) alignment to qualitatively and quantitatively (at low ppm levels) detect various types of chemical gases and vapors. These sensors rely on ink-jet printed, chemically responsive nanoparticle alignment layers that affect the orientation of nematic liquid crystals in direct contact with the nanoparticles' surface functional groups similar to alignment layers commonly used in liquid crystal display devices.1-4. Because ink-jet printing easily creates text, images, and even complex patterns using multiple inks at the same time, our sensors will permit the simultaneous detection of several gases and vapors, toxic and non-toxic, on a single device. Unlike any other sensor platform, these sensors will display a warning to the wearer or observer as a direct result of the sensing event and without the use of electrical power.

A liquid crystal sensor for detecting hazardous or non-hazardous gases and vapors, comprising: a liquid crystal cell comprising: at least two substantially transparent substrates, a substantially transparent conductive electrode layer operatively connected to each said substrate; optionally an alignment layer, independently, located on at least a portion of said electrode layers, a plurality of nanoparticles located on said alignment layer or said electrode layer, or both, said nanoparticles being generally covered by one or more ligands, and said ligands being capable of selectively chemically reacting with one or more hazardous or non-hazardous gases, and wherein a liquid crystal material is located between said substantially transparent substrates and is in contact with said ligand coated nanoparticles.

A method for forming a liquid crystal cell capable of detecting a hazardous or a non-hazardous gas or vapor, comprising the steps of: obtaining a nanoparticle composition wherein said nanoparticles are substantially covered with one or more hazardous and/or non-hazardous gas or vapor detection ligands, and a solvent; and printing at least one layer of the nanoparticle composition on one or more portions of a liquid crystal cell surface with a printer.

A hazardous or non-hazardous gas or vapor detection ink-jet printable solution comprising: a plurality of nanoparticles having a particle size of from about 0.5 to about 20 nanometers, a ligand detecting hazardous or non-hazardous coating on said nanoparticles, and a solvent; said nanoparticle ligand solution, independently, having a viscosity and a surface tension that is within 25% of that of selected ink-jet printer.

### DESCRIPTION OF THE DRAWINGS

The invention will be better understood and other features and advantages will become apparent by reading the detailed description of the invention, taken together with the drawings, wherein:

FIG. 1 shows two different overall device architectures depending on the final alignment induced by the ink-jet printed Au nanoparticle alignment layers after exposure to the gas or vapor. In FIG. 1A, the nanoparticle induces the shown homeotropic alignment (anchoring) of the nematic liquid crystals (90°), which changed in response to the sensed gas or vapor from planar (or at least a pre-tilt much lower than 90°) that can easily be detected by reduced light transmission, a change in birefringence, Δn, between crossed polarizers (see optical output above figure), or measured electrooptically in conjunction with our earlier simulation data). FIG. 1B represents the case where the initial alignment was homeotropic and changed to planar after exposure to the gas or vapor. Note the use of either a planar or a homeotropic alignment layer on the bottom substrates and the patterned optical output above the indication of bright or dark areas above the schematic of the device;

FIG. 2 shows Au nanoparticle-based liquid crystal (LC) alignment sensor systems for aliphatic amines and oxidative gases such as chlorine, bromine as well as iodine vapors. Each sensor comes with a specially designed blind probe that is not affected by the toxic species (Au NP_{blind}). The core of each nanoparticle is color-coded for the expected color change (i.e. shift in SPR band) or after sintering to solid gold. A more selective version of Au NP1 takes advantage of our silane-conjugation (Au NP1ₛₑₗ).

FIG. 3 shows Au nanoparticle (NP)-based liquid crystal alignment sensor systems for phosgene, and hydrogen cyanide. Both Au NP_{blind} and Au NP2_{blind} are designed induces either planar (the polar phosphatidylcholine bilayer-capped Au nanoparticles, Au NP2_{blind}) or vertical (homeotropic) alignment, and both are not affected by either toxic chemical. The core of each nanoparticle is color-coded for the expected color change (i.e. shift in SPR band). A more selective version of Au NP3 takes advantage of our silane-conjugation (Au NP3ₛₑₗ);

FIG. 4 shows Au nanoparticle-based liquid crystal alignment sensor system for acetone. The formation of the oxazolidinone ring is accompanied by a shift in the SPR band to longer wavelength and a change in nematic liquid crystal anchoring due to presence of methyl groups, ultimately allowing for multiple detection modes;

FIG. 5 shows different number of Au nanoparticle layers printed on rubbed polyimide coated ITO-glass: (a) more layers and (b) fewer layers. The number of layers and local nanoparticle density affects the pretilt as shown here by a varying birefringence of the ˜80 micron printed dots, viewed between crossed polarizers;

FIG. 6 shows (a) Test cell with entire ITO-area covered with Au nanoparticles inducing homeotropic alignment (crossed polarizers), (b) simulated effect of pre-tilt on threshold voltage;

FIG. 7 shows the use of the Gabriel synthesis for the design of a hydrazine sensor. The nanoparticles capped with an N-alkylated phthalimide and bound to the nanoparticle surface with an aliphatic thiol linker induce homeotropic alignment. After reaction with hydrazine gas/vapor homogeneous (planar) alignment is induced.

FIG. 8 shows (a) Dimatrix printer (LCI cleanroom), (b) zoom-in: printer cartridge, (c) camera shows nanoparticle ink-jetting, and (d) FIB-SEM image of 3-layer nanoparticle features;

FIG. 9 shows a dual sensor for the simultaneous optical sensing of two toxic gases—the sensor features an integrated duplicate row for reproducibility and a blind probe for negative/positive control. Au NP1 senses HCN, Au NP3 senses phosgene, and Au NP_{blind} always induces homeotropic alignment and is not affected by either toxic chemical. An alternative, faster response version will make use of transparent, gas permeable membranes as the bottom substrate and allows for additional selectivity by permitting only gas molecules with a specific size contact to the printed nanoparticle pattern. The thickness of the nematic liquid crystal film can be varied, but will be on the order of ˜5 to 20 μm. Analytical sensors (dose×time) have one specific opening only. Both image and analytical sensor can be constructed using gas-permeable membranes and be interfaced to a light source (OLED) as well as printed, patterned organic photo-detectors (OPDs)—Iisicon®—each provided by Merck KGaA;

FIG. 10 shows (a) printed sensor. (b & c) testing of Cl₂ and COCl₂ sensors in desiccator or glove box. (d & e) anticipated placement of sensors on visor of firefighter helmet: (d) simulated view through visor, (Bottom) POM (crossed polarizer) images of working prototypes for some toxic gases (exposure time 15 s); and

FIG. 11 shows (left) a sensor before exposure to Cl₂ gas (fully homeotropic), (middle) after exposure to Cl₂ gas (skull pattern emerges), and (right) demonstrating the contrast on a firefighter's uniform.

### DETAILED DESCRIPTION OF THE INVENTION

The gas and vapor sensors of the present invention contain liquid crystal cells such as shown in FIG. 1 wherein the liquid crystal cells contain a substantially transparent substrate such as glass, quartz, or a polymer such as polyethylene terephthalate, polycarbonate, polyacrylate, or other transparent polymers, or any combination thereof. A conductive electrode layer is contained thereon that can be made from conventional and known substantially transparent compounds such as indium oxide, tin oxide, or indium tin oxide, and the like, or any combination thereof. The term “substantially transparent” means that at least about 80%, desirably at least about 90% and preferably at least about 95% of light incident thereon is transmitted through said substrate or said electrode layer. The thickness of such a conductive electrode layer is generally from about 5 to about 200 nm (nanometers). An alignment layer is contained on the conductive electrode layer and the same is also known to the art and to the literature. Suitable compounds include polyimide, polyvinyl alcohol, SiOₓ, other polymer or aliphatic siloxane alignment layers, and the like, or any combination thereof. The thickness of the alignment layer can generally vary from about 50 to about 500 nm. In some embodiments, the alignment layer can be treated by rubbing to impart a substantially homogenous molecular orientation to the liquid crystal material prior to an electric field being applied to the cell.

In accordance with the present invention, a layer of nanoparticles is applied to the alignment layer as by printing or spray painting. Such layers comprise metal nanoparticles of gold, silver, platinum, or palladium or non-metallic nanoparticles such as carbon dots, or any combination thereof. The average diameter size of such particles is important and generally ranges from about 1 to about 20 nm, and desirably from about 1 to about 10 nm. This determination was made by transmission electron microscopy (TEM) image analysis.

An important aspect of the present invention is utilizing specific types of ligands that adhere to the nanoparticle surface by a chemical bond; either coordinative bonding, ligand to metal coordination, or covalent bonding. A key aspect in the selection of various ligands to detect specific gases such as halogens, phosgene, etc. is that they do not chemically react with the associated gas, which is to be detected. The one or more ligands at least partially or substantially cover the one or more nanoparticles, as for example at least about 60%, or about 80%; desirably at least about 90%, and preferably at least about 95%; or the entire (i.e. total) nanoparticle surface area. Ligands are selected by specific chemical reactions that result in a change in surface environment at the interface to the liquid crystal molecules. This change in surface environment leads to a change in liquid crystal alignment and as a consequence a change in light transmission between crossed polarizers. Non-hazardous gases and vapors include acetone, other ketones, and the like. Examples of hazardous gases include halogens comprising chlorine, bromine, or iodine, cyanide such as hydrogen cyanide, phosgene, aliphatic amines, dimethyl sulfide and dimethyl selenium, hydrazine, or non-hazardous gases such as ketones including chalogenides, or any combination thereof.

Aliphatic amines are a common class of toxic industrial compounds that are highly volatile allowing them to be easily released into the atmosphere. Amines are frequently used in the chemical industry and are easily absorbed through skin augmenting their toxic effects in various body tissues (acute toxicity levels: LD₅₀>2100 mg m⁻³). Chlorine, a strong oxidant, and phosgene (COCl₂), a reactive acid chloride, are both choking agents with a history of use in chemical warfare; chlorine being used as recently as the war in Syria with a string of attacks just in the last two years (onset toxic pneumonitis at 40-60 ppm). Both are frequently used in the chemical industry and are easily attainable. Phosgene, an insidious poison (toxic dose: ≥30 ppm·min), is very hazardous owing to an unpredictable asymptomatic latent phase preceding the onset of life-threatening pulmonary edema. It poses significant risks for firefighters in the vicinity of fires involving phosgene as a combustion product of Freon (often a mixture of chlorofluorocarbons or CFCs and hydrofluorocarbons) refrigeration equipment or leaks, or while fighting fires using chlorine-based halons or halotrons (liquid streaming or gaseous flooding agents used to prevent the spread of fires). A particularly tragic example of phosgene related firefighter deaths was during and after 9/11 in the fires of the twin towers. Hydrogen cyanide (HCN), a classified blood agent, is used in industrial processes such as plastics manufacturing, metal plating, and increasingly in gold mining. HCN is extremely toxic at very low levels as it is absorbed into the blood suppressing oxygen transportation (lethal airborne concentration: 180 ppm; onset of severe symptoms: 25-75 ppm). To monitor disease progression or recession, we will additionally focus on acetone, which is detectable in the breath of diabetics (ketoacidosis: 80-1,200 ppm), other ketones as well as dimethyl sulfide and dimethyl selenide occurring in the breath of patients with certain types of liver disease, and VOCs as indicators for the progression of some types of cancer.

Precision nanoparticle ink-jet printing to pattern surface-functionalized metal nanoparticles will affect the alignment of chemically inert nematic liquid crystals upon exposure to hazardous or non-hazardous gases and vapors to fabricate multi-functional sensors that can detect multiple of these gases either alone or simultaneously at low ppm levels. Ink-jet printing approach developed in our laboratory can reach resolutions up to 850 dpi (dots per inch) enables us to print and assemble multiple sensors on a single device that will permit a simple visual read-out (i.e. a warning) that one or several of these gases and vapors are present in the surrounding atmosphere or in the breath of patients. Advanced generation of these sensors include a quantitative version that will allow the wearer or user to trace and measure extended exposure (dose×time) to lower, non-fatal concentrations of these hazardous chemicals over time. A demonstrated printing resolution of 850 dpi and a feature size as small as 30-80 μm, of very small sensors can be integrated into unmanned robotic vehicles or drones coupled with an electronic read-out. The resulting sensors placed between two crossed polarizers would be illuminated by a small light source and read by patterned photo-detectors on the opposite side of the light source to detect the passage of light through the sensing cell. The presence or absence of light, or reduction of intensity, will determine whether a chemical agent is present or not. A reflective device will be used at lower resolution (larger sensor) for direct read-out by a human wearer.

The sensors will be simple to operate, and rather than paired to a visual output or display that consumes power, the sensors will be the display that is (1) easily interpreted by the operator because an image and/or text appears in the event of exposure to specific gases and vapors, (2) additionally generates an electro-optic signal that can yield a numerical value reflecting the amount (or dose×time) of the gas or vapor present in the environment or breath, and (3) can give off an electronic signal or colorimetric response (by coupling to a photo-detector array) that is resistant to potential optical interference. Multi-readout potential will alleviate significant drawbacks of other wearable (mobile) sensors that always need power or are solely based on a colorimetric response. One or some forms of color blindness affects one in twelve men (8%), and men are the majority among emergency responders, military personnel and firefighters.

We firmly established that the surface chemistry of metal nanoparticles and quantum dots is the decisive factor for attaining either planar or vertical alignment in nematic liquid crystals. Nanoparticles with capping ligands featuring aliphatic chains induce vertical alignment. Ligands with polar (ionic) functional groups such as carboxylic acid groups in thioglycolic acid capped CdTe quantum dots or L-cysteine-capped Au nanoparticles do not alter the alignment of nematic liquid crystals and planar alignment is retained.

In principle, any design and size of the vertical or planar alignment domains is possible via ink-jet printed nanoparticles, including the printing of logos, text, and larger panels for example for bigger warning signs in industrial settings.

The ligands are selectively sensitive to generally only one of said types of gases. Thus, sensors can be made that detect only one type of gas such as hydrogen cyanide as utilized in the mining of gold. Also, various ligands can be utilized that detect chlorine, cyanide, or phosgene, which are (or were recently) used as chemical weapons). In this regard the sensors protect military personnel and first responders in conflict zones.

The following is a list of suitable ligands that can be utilized in the present invention as well as the type of gas they selectively detect. Strong oxidizing gases such as halogens (Cl₂, Br₂ and I₂) can be detected with metal nanoparticles capped (covered) with thiols, aliphatic or otherwise, (i.e. non-aliphatic) with the length of the aliphatic group being from about C1 to about C20, desirably from about C2 to C15, and preferably from about C6 to about C12. Non-aliphatic compounds or aromatic thiols can have from about 2 to about 12 carbon atoms, and preferably from about 2 to about 6 carbon atoms. To avoid false negatives for multi-gas sensors, specific thiols are used that are made via a cross-linked silane shell, which does not react to halogen gases by desorption from the nanoparticle surface.

Cyanide can be detected by nanoparticles covered by any amino acid, except for cysteine, having from about 4 to about 11 carbon atoms. Also, thioglycolic acid, or a cysteine ((D), (L), or DL-), or an aliphatic thiol having an (omega) ω-carboxylic acid group (see formula below) can be utilized. These particular ligand shells are for specific sensors detecting only cyanide, for example used in gold mining.

[chemical structure]

The number of repeat groups, i.e. n can be 1, or 2 to about 16, or preferably from about 10 to about 16. Still another ligand is an aliphatic thiol having an omega-amino group having the formula

[chemical structure]

wherein n is 0, or 1 to about 10, and preferably from about 0 to about 2.

Phosgene can be detected with cysteine ((D), (L), or DL-), or with aliphatic thiols with an ω-amino group having the formula:

[chemical structure]

where n is 0, or from 1 to about 10, and preferably is from about 0 to about 2.

Aliphatic amines can be detected using nanoparticles with ω-carboxylic acid substituted aliphatic thiol ligands bound to the nanoparticle surface with the carboxylic acid group that is available for salt formation with the toxic amines. The aliphatic group is a hydrocarbon chain with a terminal thiol group. The aliphatic thiol terminal carboxylic acid group has the formula

[chemical structure]

where n is 0, or 1, or 2 to about 16 with from about 10 to about 16 carbon atoms being preferred.

Ketones such as acetone can be detected with the above-mentioned cysteine-capped (covered) nanoparticles, where the NP surface is simultaneously capped (covered) with thioglycolic acid (ratio cysteine/thioglycolic acid of about 100 to about 1 and preferably from about 10 to 1 to about 1 to 1).

Dialkylchalcogenides can be detected by nanoparticles capped (covered) with weak-binding ligands such as an amino acid (except cysteine), or an aliphatic amine having from C1 to C20, and preferably from about 6 to about 12 carbon atoms, or citric acid. Varying degrees in induced pre-tilt can be used to distinguish these.

Hydrazine can be detected by the reaction with alkylated phthalimides linked to the nanoparticle surface with the help of an aliphatic hydrocarbon having from 1 to about 12 C atoms covalently bound to the aromatic benzene ring and featuring a substitution at the end that facilitates bonding to the nanoparticle surface. The alkylation species is a primary aliphatic amine with varying chain length (from C1 to about C20) with from about C6 to about C12 being preferred.

Both dimethyl sulfide (Me₂S) and dimethyl selenide (Me₂Se) can be sensed with metal nanoparticles that are initially covered with weaker binding ligands such as amino acids (e.g., lysine), aliphatic amines having from about C1 to about C20, and preferably from about C6 to about C12 atoms, and citric acid, with citric acid being preferred. Each dimethyl chalcogenide binds to Au and Ag nanoparticle surfaces, but selectivity towards one or the other might be difficult to achieve. Selectivity would be especially critical since both are important in the monitoring of some types of liver disease, where progression is indicated by an increase in Me₂S and a decrease in Me₂Se over time. For both Me₂S- and Me₂Se-capped metal nanoparticles a change of nematic liquid crystal alignment from planar for the initially citric acid-capped Au nanoparticles to homeotropic (or higher pretilt) is expected. It is possible however that the degree of induced pre-tilt is different for each of the dimethyl chalcogenides, which we will carefully test using optical and electro-optical measurements. Even the smallest difference in pre-tilt of the nematic liquid crystal alters light transmission and results in a noticeable change in birefringence when crossed polarizers are used as shown in FIG. 1. Changes have been observed in birefringence for some thiol-capped Au nanoparticles when too few and a varying number of nanoparticle layers were printed to induce fully homeotropic alignment (FIG. 5). This difference in induced pre-tilt could be the result of the difference in packing of each of the dimethyl chalcogenide ligands on the nanoparticle surface. If so, the ratio between the two ligands, when monitoring a given person (patient) periodically, could then be used to assess an increase or decrease in concentration for either one of them.

It is also within the scope of the present invention to utilize ligands that actually block out the detection of undesired gases so that the sensors of the present invention only detect a desired gas, such as chlorine gas or cyanide. In other words, only a desired type of gas can be detected by the sensors of the present invention within a specific type of gaseous environment. For example, gold mining uses cyanide, which would be the primary toxic species to be detected. Aliphatic amines would be the primary toxic species in meat processing plants and chemical manufacturing of these chemicals. Chlorine and phosgene are prime examples of toxic gases occurring during fire involving refrigeration (i.e. air conditioning) units or leaks or using halons or halotrons (liquid streaming or gaseous flooding agents used to prevent the spread of fires) to extinguish fires. Chlorine, phosgene and cyanide have been used in chemical warfare and can selectively been detected by the sensors with specific (selective) nanoparticle ligands for each gas.

The type of liquid crystals that are utilized in the sensors of the present invention are generally nematic liquid crystals since (i) nematic liquid crystals are available as non-reactive, chemically inert materials and widely used in display industry. The number of nematic liquid crystals is large and is known to the art and to the literature, Examples of some suitable nematic liquid crystals include fluorinated and chemically inert nematic liquid crystals with appropriate transition temperatures. More specifically, a key parameter for the utilization of the various types of liquid crystals to be used as singles, i.e. only one liquid crystal, but more likely and preferably in mixtures, i.e. two or more liquid crystals, is that the liquid crystals should be non-reactive towards the toxic gas or vapor being detected. Moreover, they should not have any functional group that will bind to gold or other nano type metals such as silver, platinum and palladium. With respect to specific liquid crystals, a single type of liquid crystal, i.e. 5CB is a single crystal available from various suppliers including Sigma Aldrich, Synthon GmbH, Merck. Another single liquid crystal is Felix-2900-03 from Merck. Suitable liquid crystal mixtures include TL203, MLC-6610 and MLC-2169 (all from Merck) to name just a few examples, as well as other liquid crystals that are proprietary.

A distinct advantage of the sensors of the present invention, inasmuch as they are specifically orientated to the detection of a particular type of gas or vapor, is that they be utilized for the detection of other different and distinct types of gases or vapors simply by removing the above-described liquid crystal cell such as shown in FIG. 1 and inserting a different liquid crystal cell designed to specifically detect a different type of gas or vapor. Such interchangeability adds to the desired use of the sensors of the present invention.

like.

Some of the ligands capping the nanoparticle surface induce planar, others homeotropic alignment. To achieve a visual readout for each type of sensor, two different overall device architectures exist, i.e. see FIG. 1. The use of planar and/or homeotropic polymer or SiOₓ alignment layers on both substrates fulfills two essential requirements. First, research has shown that more defined and homogeneous droplets of the nanoparticle ink form after jetting from the piezoelectric printer head on such treated substrates in comparison to plain glass. Second, degenerate alignment is prevented on the opposite substrate, which leads overall to higher and more homogeneous contrast i.e. better readability of the sensor device. This also allows the printing of functionalized nanoparticles on only one of the two substrates, which avoids the use of more of the precious nanoparticles than necessary and eliminates the need to tediously align the patterns from top and bottom substrate in the device assembly step.

### EXAMPLES

Synthesis and Characterization of Requisite Au Nanoparticles with Ligand.

The synthesis of specific Au nanoparticles will be used as examples that serve as proof-of-concept for the specific detection of four hazardous chemical gases from four distinct chemical classes, aliphatic amines (RNH₂), hydrogen cyanide (HCN), chlorine (Cl₂) and phosgene (COCl₂). In light of their immensely toxic and reactive nature, and considering recent horrific events of their misuse or occurrence in tragedies, systems will be used.

The specific surface chemistries of the Au nanoparticles (core diameter ranging from 1.5 to ˜10 nm or ˜20 nm) modified with a ligand were chosen to sense these four toxic gases are shown in FIGS. 2, 3, and 11. The synthetic schematics also depict the respective liquid crystal alignment modes expected from the change in surface chemistry after reaction with the toxic gases or vapors. Each set additionally shows a chemically resistant (inert), non-reactive control nanoparticle that will serve as blind control in the final sensors. As indicated with the color coding of the nanoparticle cores in these figures, some reactions with the hazardous gases or vapors do not only affect the nanoparticle surface chemistry, they induce aggregation of the Au nanoparticles, which in turn results in a shift of the surface plasmon resonance band wavelength, allowing for an additional signal that can be read or measured (i.e. a change in color due to a change in the surface plasmon resonance of the nanoparticle once aggregated).

All synthesized nanoparticles undergo rigorous characterization. After purification by a series of washing, centrifugation and re-precipitation steps all Au nanoparticles are routinely analyzed by ¹H NMR, where the resulting spectra allow us to determine that no free, unbound ligands are present. To determine surface coverage, we then preformed I₂ decomposition (where the oxidative potential of the I₂ vapor is used to oxidize the thiols to disulfides, leaving bare nanoparticles that sinter to form solid gold—the same reaction used for the Cl₂ gas sensor; Au NP2 in FIG. 2). ¹H NMR of the soluble residue from this decomposition reaction is used together with an internal standard (precise amount of a soluble inert organic compound with characteristic proton chemical shifts) to determine the required amount of ligands, which then is corroborated with thermogravimetric analysis (TGA). UV-vis absorption spectra were recorded to determine wavelength and intensity of the surface plasmon resonance (SPR) band, which provides first clues about the diameter of the nanoparticle core. Finally, we characterize the nanoparticles with transmission electron microscopy (TEM) and dynamic light scattering to measure size, size distribution, and shape. After TEM image analysis, we used our established geometric algorithm to precisely determine composition and ligand coverage.

Handling of the hazardous halogen gases and amine vapors is not problematic as each of these is routinely used in organic as well as inorganic syntheses in many laboratories. We have established that carboxylic acid end-capped nanoparticles induce planar alignment (anchoring) of nematic liquid crystals and predict that coordination of aliphatic amines will result, as observed for aliphatic thiols, in a change to homeotropic alignment (Au NP1).

We know from experience working with silane-conjugated thiol-capped Au nanoparticles such as Au NP_{blind} blind that the three-dimensional, condensed polysiloxane shell is chemically inert to the oxidizing ability of halogen gases, which is why these were chosen as blind control.

For the sensing of hydrogen cyanide and phosgene, the sensing strategy starts with the synthesis of Au nanoparticles featuring polar, hydrophilic ligands such as cysteine or 1,ω-mercapto carboxylic acids. Both types of Au nanoparticles induce planar alignment of nematic liquid crystals as shown earlier in FIG. 9 (center). Here Au NP_{blind} or Au NP2_{blind}, with the latter reportedly inert to cyanide etch, can be used as blind control (FIG. 3).

Cyanide and phosgene were handled with extra care in comparison to the two toxic chemicals described earlier (amines and halogens), but are known to be used for a variety of organic synthesis such as the cyanohydrin reaction for CN⁻ (using NaCN) and various reactions with practically all types of nucleophiles (N⁻, O⁻, S⁻) for phosgene. Phosgene is commercially available from chemical suppliers such as Sigma Aldrich as 20% solutions in toluene. Safe handling of these chemicals was ensured by performing all sensor tests in a glove box with nitrogen (inert) gas feeds and vents including wash bottles containing reagents that safely react with cyanide or phosgene to harmless products that can be safely added to our regular laboratory chemical waste.

To sense acetone vapor, for example in the breath of diabetics, we employ a recently published chemical transformation of Au nanoparticles initially capped with cysteine and, after reaction with acetone, capped with oxazolidin-5-one (FIG. 4). This chemical transformation on the nanoparticle surface was accompanied by a color change from purple to blue (i.e. shift of the SPR band to longer wavelengths). Moreover and critical for the proposed sensor concept, it is believed that a clearly discernable change in nematic liquid crystal alignment from planar to homeotropic (or at least much larger pre-tilt) based on the presence of aliphatic methyl groups on the surface of the Au nanoparticles after ring closure to the oxazolidinone.

Preparation of Initial Test Surfaces

Before printing the Au nanoparticles for multiresponsive liquid crystal sensors, each type of Au nanoparticle is deposited on ITO-glass with polymer or SiOₓ (planar and homeotropic) alignment layers. We used spin coating on one of the two substrates in such way that the entire field-addressed area (the area where top and bottom ITO overlap) was covered with functionalized Au nanoparticles. The substrates were then characterized by high-resolution focused ion beam scanning electron microscopy (FIB-SEM) to determine the thickness of the nanoparticle layer. Test cells made in this way were filled with chemically inert nematic liquid crystal mixtures as shown in FIG. 6a.

For each cell, the liquid crystal alignment, pre-tilt, and anchoring energy was determined. We measured the polar-anchoring energy using Yokoyama-van Sprang's method, enhanced by Lavrentovich et al., and expanded to the homeotropic case by Wu et al., which is based on the measurement of the optical phase retardation as a function of applied voltage. For aliphatic thiol-capped Au nanoparticles we obtained a value of 6.8×10⁻⁴ J m⁻², which is similar in magnitude to commercially available polymer-based homeotropic alignment layers. We measured the electro-optic response and use our simulation data (FIG. 6b) to assess the effect of varying pre-tilt. The same set of measurements was then be repeated after exposure of the test sensor to different concentrations (varied by exposure time) to the various gases and vapors. While in most cases a clearly noticeable change in light transmission and birefringence between crossed polarizers is observed, in some cases, the described more careful assessment of the optical and electro-optic response is needed, especially when a quantitative detection of these gases and vapors is essential. Finally, we extended the detection of said gases to absorption spectroscopy mostly in the visible portion of the electromagnetic spectrum to detect shifts in the surface plasmon resonance wavelength of the Au nanoparticles after exposure to the gases and vapors of interest.

Ink-jet printed nanoparticle patterning, device assembly, and testing.

The last step of the preparation of the patterned nanoparticle-liquid crystal sensing devices via inkjet printing of the functionalized NPs and integration into patterned hybrid aligned liquid crystal cells (see FIG. 1). For printing, a desktop material ink-jet printer Fujifilm Dimatrix DMP-2800 (Santa Clara, Calif.), or any other model or materials ink-jet printer, and appropriate cartridges has used (FIG. 8a, b). The nanoparticle solution was prepared using a suitable solvent is mildly sonicated in a standard ultrasonic water bath for 1 min before filling the printer cartridges. An important aspect of the present invention is the formulation of pre-ink solutions having a surface tension and viscosity that is similar or compatible to that of the ink-jet printer. That is, the formulated pre-ink solutions must be of a range that matches the viscosity and surface tension requirements utilized by the ink-jet printer. In other words, the nano-ink solution must conform to the requirements of the piezo-based nozzles of the printer cartridges. Suitable viscosities of the nano-ink with respect to the above-noted printer, is in the range of from about 6 to about 16, desirably from about 8 to about 14, and preferably to about 10 to 12 cPs. The surface tension of the nano-ink is in the range of from about 28 to about 42 dynes cm⁻¹ or desirably from about 30 to about 40. For most nanoparticles, o-xylene is the most suitable solvent, showing viscosities around 10 cPs and values for the surface tension at around 35 dynes cm−1 at nanoparticle concentrations of around 35 mg mL⁻¹. Since many different types of ink-jet printers with specifically designed cartridges exist, they vary with respect to viscosity and surface tension, thus, the overall viscosity range of many material ink-jet printers is from about 5 to about 20 and the overall surface tension range is from about 20 to about 50 dynes/cm. The viscosity and surface tension of the nanoparticle ligand solution of the present invention should be within about 30%, desirably about 20%, and preferably within 10% of the ink-jet printer recommended values. Other suitable solvents include water-alkyl alcohol mixtures, or water-glycerol, or water-ethylene glycol solvent mixtures wherein the alkyl alcohol has from 1 to about 6 carbon atoms with methanol and ethylene glycol being preferred. Also, any combination of the above-noted solvents can be used. Jetting waveforms are adjusted to provide jetting with the optimal characteristics for each of the prepared inks (FIG. 8c). For hydrophilic nanoparticles, in case o-xylene does not sufficiently dissolve them, other high viscosity solvents were used, and aqueous glycols appear here as an appropriate first choice. The thickness of the ink-jet printed nanoparticle layer was measured by high-resolution FIB-SEM (FIG. 8d), which allows us to calculate how many nanoparticle layers were printed considering the nanoparticle concentration and average size determined from TEM image analysis (outlined above). Sensors were assembled by filling cells with chemically inert nematic liquid crystal mixtures featuring high thermal stability to allow for a wide operating temperature range of the final sensors. Mixtures are provided with phase stabilities for specific applications such as military or firefighters. Stated in different terms, the preparation of the nano-ink involves a specific concentration of the specific nanoparticle and the solvent (or solvent mixture). The concentration of the particles was adjusted to reach the best possible values (preferred) for viscosity and surface tension. This is favorable because it delivers the best possible printing results. For spray painting, the requirements on the nano-ink are less stringent, but the patterns are less sharp and less homogeneous. We have successfully printed on glass coated with: SiOx, polyimides favoring planar or homeotropic anchoring—various types, polyvinyl alcohol, and just indium tin oxide (ITO). We have also successfully printed on flexible polymer films coated with ITO and alignment layers (as those mentioned above). Generally, any type of indicia can be printed on the liquid crystal cell including symbols, patterns, designs, logos, displays, pictures, characters, and so forth, or any combination thereof.

As outlined in “Preparation of Initial Test Surfaces” we then tested the response of the liquid crystal alignment sensors to each of the target agents. This includes determination of response time, minimum detection limit, effects of interferants, and effects of temperature and humidity.

The complete schematic design of a dual gas/vapor sensor is shown in FIG. 9. The top and bottom row are identical and printed with three different types of Au nanoparticles; NP1 sensing one airborne toxic chemical, NP2 sensing another one, and NP_{blind} (a silanized alkylthiol-capped Au NP) serving as the blind control to allow the observer to be assured that the device is functioning properly. Here Au NP_{blind} blind is not affected by any chemical exposure with reactive species because the siloxane shell renders this particle not only thermally but also chemically highly stable against surface chemical reactions. In turn, this printed domain is not affected and the alignment does not change upon exposure to the hazardous vapor or gas. The bottom row is a duplicate of the first, i.e. each sensing event is run autonomously in duplicate to make sure the observer does not get a false or incomplete reading.

With available printing resolutions ranging from 340 to 850 dpi, more than a dual design on the same sensor is easily possible and printed feature sizes can be as small as 30 μm if the smaller nozzles on the cartridge are chosen (1 pL cartridge). Considering the use for specific applications, some of the hazardous species can be sensed individually in the presence of a non-reactive control nanoparticle and not require a dual or multiplexed sensor. Aside from single component nematic liquid crystals, we have successfully tested several wide-temperature range nematic liquid crystal mixtures for nanoparticle-induced alignment patterning (e.g., TL203 from DIC Japan and MLC-6610 from Merck), and the hazardous gases or vapors did not affect these.

Finally, we incorporated flexible, gas-permeable membranes as top or bottom layers of these devices, since we have demonstrated that we can print on flexible ITO-coated polymer substrates as well. A key requirement of the various membranes is that they be non-reactive towards the toxic gases and vapors that are trying to be detected. The membranes tested were poly(trimethylsilylnorbornene) (PTMSN)—a polymer with a rather high porosity and gas permeability (excellent for larger gaseous molecules such as aliphatic amines or phosgene). Other, less porous, transparent and chemically inert polymer membranes were made of poly(dimethylsiloxane) (PDMS) or Nafion®. Such flexible substrates enhance the utility of the sensing devices (wearable sensors) and allow us to build more selective qualitative sensors with faster response times. The penetration of gases from the side of glass cells in similar sensor devices, showed that a small headspace of air between top substrate and nematic liquid crystal are feasible solutions to detect gases (e.g., H2S) at low ppm levels. However, the lower diffusivity of larger gas or vapor molecules of about 1·10⁻⁶ cm² s⁻¹ through the nematic liquid crystal is be too low for a fast response sensor. The gas permeable membranes eliminated the issue of low diffusivity particularly for larger gas and vapor molecules by allowing direct contact with the reactive nanoparticles.

Photographs of sensor prototypes are shown in FIG. 10. The gases tested were Cl₂, COCl₂, CN—, and various amines. We also used an airbrush-stencil technique to pattern reactive nanoparticle alignment layers, and the sensors performed equally well in tests with the mentioned toxic gases or vapors.

While in accordance with the Patent Statutes, the best mode and preferred embodiments have been set forth, the scope of the invention is not limited thereto, but rather by the scope of the attached claims.

What is claimed:

1. A liquid crystal sensor for detecting hazardous or non-hazardous gases and vapors, comprising:
a liquid crystal cell comprising: nematic liquid crystal molecules; at least two substantially transparent substrates, a substantially transparent conductive electrode layer operatively connected to each said substrate; optionally an alignment layer, independently, located on at least a portion of said electrode layers, a plurality of nanoparticles located on said alignment layer or said electrode layer, or both, said nanoparticles being substantially covered by one or more ligands, and said ligands being capable of detecting hazardous, or non-hazardous gases or vapors; and
wherein said nematic liquid crystal molecules are located between said substantially transparent substrates and are in contact with said ligand coated nanoparticles.

2. The liquid crystal sensor according to claim 1, wherein said nanoparticles comprise one or more of silver, gold, palladium, platinum, or carbon dot cores, or any combination thereof; wherein the particle size of said nanoparticles is from about 1 to about 20 nanometers; wherein at least about 60% of the total surface area of said nanoparticles are coated with said one or more ligands, and optionally wherein said printed nanoparticles are in the form of a pattern, a symbol, a design, a logo, a display, a picture, a character, or any combination thereof, and wherein said printed matter is on said electrode layer, or said alignment layer, or any combination thereof.

3. The liquid crystal sensor according to claim 2, said ligands being capable of chemically reacting with said one or more hazardous or non-hazardous gases, or vapors, or any combination thereof; wherein said hazardous gases comprise a halogen; a phosgene, a cyanide, an aliphatic amine, a hydrazine, dimethyl sulfide or dimethyl selenium, or any combination thereof; wherein said non-hazardous gas comprises a ketone or a dialkylchalcogenide; and wherein at least about 80% of the total surface area of the nanoparticles is coated with said one or more ligands.

4. The liquid crystal sensor according to claim 3, wherein said substantially transparent substrate comprises glass, quartz, or a substantially transparent polymer, or any combination thereof; wherein said substantially transparent conductive electrode comprises indium tin oxide, tin oxide, or indium oxide, or any combination thereof; and wherein said alignment layer comprises a polyimide, polyvinyl alcohol, SiOₓ where x is 0 to 2, or an aliphatic siloxane; or any combination thereof, and wherein said liquid crystal cell has a pattern printed thereon.

5. The liquid crystal sensor according to claim 2, wherein said hazardous gas is chlorine, iodine, or bromine, or any combination thereof; and wherein said ligand is an aliphatic thiol wherein said aliphatic group has from about 1 to about 20 carbon atoms, or a non-aliphatic thiol having from about 2 to about 12 carbon atoms.

6. The liquid crystal sensor according to claim 3, wherein said hazardous gas is chlorine, iodine, or bromine, or any combination thereof; or wherein said ligand is an aliphatic thiol wherein said aliphatic group has from about 6 to about 12 carbon atoms.

7. The liquid crystal sensor according to claim 2, wherein said hazardous gas is hydrogen cyanide, wherein said one or more ligands cover at least about 80% of the total surface area of said nanoparticles covering said nanoparticle surface, and wherein said ligand is an amino acid, except for cysteine, having a total of from about 4 to about 11 carbon atoms; or a thioglycolic acid; or cysteine (D), (L), or (DL-), or an aliphatic thiol having a carboxylic acid group having the formula [chemical structure]
where n is from 1 or 2 to about 16, or an aliphatic thiol having an omega-amino group having the formula [chemical structure]
wherein n is 0, or 1 to about 10, or any combination thereof.

8. The liquid crystal sensor according to claim 3, wherein said hazardous gas is hydrogen cyanide; and wherein said ligand is an aliphatic thiol having a carboxylic acid group having the formula [chemical structure]
where n is from about 10 to about 16.

9. The liquid crystal sensor according to claim 2, wherein said hazardous gas is phosgene; and wherein said ligand is a cysteine (D), (L), or (DL-); or an aliphatic thiol having an omega-amino group wherein said aliphatic thiol comprises [chemical structure]
wherein n is 0, or 1 to about 10.

10. The liquid crystal sensor according to claim 3, wherein said hazardous gas is phosgene; and wherein said ligand is a cysteine (D), (L), or (DL-); or an aliphatic thiol having an amino group having the formula [chemical structure]
wherein n is from about 0 to about 2.

11. The liquid crystal sensor according to claim 2, wherein said hazardous gas is an aliphatic amine; and wherein said ligand is an omega-carboxylic acid substituted aliphatic thiol having the formula [chemical structure]
wherein n is 0, or 1, or 2 to about 16.

12. The liquid crystal sensor according to claim 3, wherein said hazardous gas is an aliphatic amine; and wherein said ligand comprises an omega-carboxylic acid substituted aliphatic thiol with the carboxylic acid group bound to said nanoparticle surface, having the formula [chemical structure]
wherein n is from about 10 to about 16.

13. The liquid crystal sensor according to claim 2, wherein said non-hazardous gas is ketone; and wherein said ligand is a mixture of cysteine (D), (L), or (DL-) and thioglycolic acid having a ratio of cysteine/thiol glycolic acid of from about 100 to about 1.

14. The liquid crystal sensor according to claim 3, wherein said non-hazardous gas is a dialkylchalcogenide; and wherein said ligand is an amino acid, or citric acid.

15. The liquid crystal sensor according to claim 2, wherein said hazardous gas is hydrazine; and wherein said ligand is an alkylated phthalimide linked to the nanoparticle surface via a hydrocarbon aliphatic having from 1 to about 12 carbon atoms, covalently bound to the aromatic benzene ring, and wherein said alkylation species is a primary aliphatic amine having from 1 to about 20 carbon atoms.

16. The liquid crystal sensor according to claim 3, wherein said hazardous gas is hydrazine; and wherein said ligand is an alkylated phthalimide linked to the nanoparticle surface via an aliphatic hydrocarbon having from about 1 to about 12 carbon atoms, that is covalently bound to the aromatic benzene ring, and wherein said alkylation species is a primary aliphatic amine having from 1 to about 20 carbon atoms.

17. The liquid crystal sensor according to claim 2, wherein said hazardous gas is dimethyl sulfide or dimethyl selenide; and wherein said ligand is a weak ligand comprising an amino acid.

18. The liquid crystal sensor according to claim 3, wherein said hazardous gas is dimethyl sulfide or dimethyl selenide; and wherein said ligand is citric acid.

19. A method for forming a liquid crystal cell capable of detecting a hazardous or a non-hazardous gas or vapor, comprising the steps of:
applying a nanoparticle composition to said liquid crystal cell wherein said nanoparticles are substantially covered with one or more hazardous and/or non-hazardous gas or vapor detection ligands, said cell having nematic liquid crystal molecules therein,
said covered ligands being capable of detecting hazardous or non-hazardous gases, or vapors, and
printing at least one layer of the nanoparticle composition on one or more portions of a liquid crystal cell surface with a printer.

20. The method according to claim 19, wherein at least about 60% of the total surface area of said nanoparticles are covered with said one or more ligands.

21. The method according to claim 19, wherein at least about 60% of the total surface area of said nanoparticles are covered with said one or more ligands; wherein said nanoparticles comprise gold, silver, platinum, palladium, or a carbon dot, or any combination thereof; wherein said liquid crystal cell comprises a substrate, an electrode layer on the surface of said substrate; and an optional alignment layer located on at least a portion of said electrode layer; wherein said nanoparticle ligand containing composition is printed on at least one or more portions of said electrode layer, and/or said alignment layer; and
wherein said ligand comprises,
an aliphatic thiol wherein said aliphatic group has from about 1 to about 20 carbon atoms; or
a non-aliphatic thiol having from about 2 to about 12 carbon atoms; or
an amino acid except for cysteine group having a total of from about 4 to about 11 carbon atoms; or
a thioglycolic acid; or
a cysteine (D), (L), or (DL-); or
an aliphatic thiol having a carboxylic acid group having the formula [chemical structure]
where n is from 1 to about 16; or
an aliphatic thiol having an amino group wherein said aliphatic thiol comprises [chemical structure]
wherein n is 0, or 1 to about 10; or
an alkylated phthalimide linked to the nanoparticle surface via an aliphatic hydrocarbon chain having from 1 to about 12 carbon atoms covalently bound to the aromatic benzene ring wherein said alkylation species is a primary amine having from about 1 to about 20 carbon atoms; or
an amino acid, an aliphatic amine having from 1 to about 20 carbon atoms, or a weak ligand, or citric acid, or any combination of said ligands.

22. The method according to claim 21, wherein said nanoparticles have a size of from about 1 to about 20 nanometers; wherein at least about 90% of the total surface of said nanoparticles are covered with said one or more ligands.

23. The method according to claim 19, wherein said nanoparticles have a size of from about 1 to about 10 nanometers; wherein said printed nanoparticles are in the form of a pattern, a symbol, a design, a logo, a display, a picture, a character, or any combination thereof, and wherein said printed matter is on said electrode layer, or said alignment layer, or a combination thereof.

24. The method according to claim 23, including ortho-xylene as a solvent.

25. The method according to claim 20, wherein the viscosity of said nanoparticle containing composition is from about 5 to about 20 cPs; and wherein the surface tension of said nanoparticle containing composition is from about 20 to about 50 dynes per centimeter.

26. The method according to claim 22, wherein the viscosity of said nanoparticle containing composition is from about 8 to about 14 cPs; and wherein the surface tension of said nanoparticle containing composition is from about 30 to about 40 dynes per centimeter.

27. The method according to claim 20, wherein said non-hazardous or said hazardous gas comprises a halogen, cyanide, phosgene, aliphatic amine, hydrazine, dimethyl sulfide or dimethyl selenium, or any combination thereof.

28. The method according to claim 22, wherein said non-hazardous or said hazardous gas comprises a halogen, cyanide, aliphatic amine, hydrazine, phosgene, dimethyl sulfide or dimethyl selenium, or any combination thereof.
